(19) United States
(12) Patent Application Publication
(10) Pub. No.: US 2015/0018360 A1
(43) Pub. Date: Jan. 15, 2015

(54) COMPOSITIONS AND METHODS FOR TREATING METABOLIC DISORDERS

(71) Applicants: Reza Halse, Boston, MA (US); Jeffrey James Roix, Boston, MA (US); Saurabh Saha, Wellesley Hills, MA (US)

(72) Inventors: Reza Halse, Boston, MA (US); Jeffrey James Roix, Boston, MA (US); Saurabh Saha, Wellesley Hills, MA (US)

(73) Assignee: BIOMED VALLEY DISCOVERIES, INC., Kansas City, MO (US)

(21) Appl. No.: 14/344,665

(22) PCT Filed: Sep. 13, 2012
(86) PCT No.: PCT/US2012/055088

§ 371 Date: Sep. 25, 2014

Related U.S. Application Data
(60) Provisional application No. 61/533,934, filed on Sep. 13, 2011.

(51) Int. Cl. A61K 31/40; A61K 31/4985

(52) U.S. Cl. CPC A61K 31/40; A61K 31/4985
USPC 514/250; 514/428

(57) ABSTRACT

The present invention provides, inter alia, compositions containing enantiomerically pure (R)(+)-amisulpride or enantiomerically pure (R)(+)-sulpiride, optionally with dopamine receptor modulators. The present invention also provides compositions containing racemic (RS)-amisulpride or (RS)-sulpiride in combination with dopamine receptor modulators. Methods for preventing, treating, or ameliorating the effects of a metabolic disorder or key element thereof, for modulating blood glucose levels, and for preventing, treating, or ameliorating the effects of diabetes in a subject are also provided. Additionally, the present invention provides methods for counter-acting the dopamine antagonist activity of (S)-amisulpride in racemic (RS)-amisulpride, or the dopamine antagonist activity of (S)-sulpiride in racemic (RS)-sulpiride, administered to a subject to prevent, treat, or ameliorate the effects of a metabolic disorder.

### CROSS-REFERENCE TO RELATED APPLICATIONS

The present invention claims benefit to U.S. provisional application Ser. No. 61/533,934 filed Sep. 13, 2011, the entire contents of which are incorporated by reference.

### FIELD OF THE INVENTION

The present invention relates, inter alia, to compositions and methods for treatment of metabolic disorders and their key elements, such as, e.g., disorders associated with glucose metabolism.

### BACKGROUND OF THE INVENTION

Several pharmacoepidemiological studies have established a risk of drug-induced diabetes in the treatment of psychiatric disorders, particularly in patients taking second generation antipsychotic (SGA) drugs (1-3). Prospective clinical studies monitoring diabetes progression with SGA treatment have presented a more complicated picture, suggesting that significant variation in risk exists with respect to the antipsychotic agent used and the preexisting metabolic state of the patient (4,5).

Amisulpride, an SGA belonging to the benzamide structural class, is a racemic drug approved for use in Europe for both the treatment of positive and negative symptoms associated with schizophrenia (6). It is prescribed in a wide dose range, with the treatment of positive symptoms requiring as high as 1200 mg/day and negative symptoms as low as 50 mg/day (6). In clinical practice, amisulpride has less impact on glucose metabolism and is often used as an alternate treatment approach in patients with metabolic syndrome resulting from SGA use. A number of studies have suggested that amisulpride has a relatively low risk of inducing weight gain and diabetes (2,7,8), although, this racemic drug often causes hyperprolactinemia, likely due to potent inhibition of dopamine D₂/D₃ receptors (9-11). Previous studies have not defined whether the improved metabolic profile of amisulpride is due to reduced diabetogenic effect or some anti-diabetic action.

### SUMMARY OF THE INVENTION

The present invention is directed to, inter alia, the glucose lowering effects of amisulpride and the decoupling of the anti-diabetic actions of amisulpride from known amisulpride effects on dopaminergic signaling. The results are particularly surprising because other molecules in this class of drugs have pro-diabetic, not anti-diabetic effects.

Accordingly, one embodiment of the present invention is a composition that comprises a pharmaceutically acceptable carrier and a therapeutically effective amount of an enantiomerically pure (R)(+)-amisulpride, an enantiomerically pure (R)(+)-sulpiride, or a pharmaceutically acceptable salt thereof.

Another embodiment of the present invention is a composition that comprises (i) a pharmaceutically acceptable carrier; (ii) a therapeutically effective amount of racemic (RS)-amisulpride, (RS)-sulpiride or a pharmaceutically acceptable salt thereof; and (iii) a dopamine receptor modulator.

Yet another embodiment of the present invention is a composition that comprises (i) a pharmaceutically acceptable carrier; (ii) a therapeutically effective amount of an enantiomerically pure (R)(+)-amisulpride, an enantiomerically pure (R)(+)-sulpiride, or a pharmaceutically acceptable salt thereof; and (iii) a dopamine receptor modulator.

An additional embodiment of this invention is a method for preventing, treating, or ameliorating the effects of a metabolic disorder or key element thereof in a subject. This method comprises administering to the subject an effective amount of any of the compositions disclosed herein.

A further embodiment of this invention is a method for modulating blood glucose levels in a subject. This method comprises administering to the subject an effective amount of any of the compositions disclosed herein.

Another embodiment of this invention is a method for preventing, treating, or ameliorating the effects of diabetes in a subject. This method comprises administering to the subject an effective amount of any of the compositions disclosed herein.

Yet another embodiment of the present invention is a method for counter-acting the dopamine antagonist activity of (S)-amisulpride in racemic (RS)-amisulpride administered to a subject to prevent, treat, or ameliorate the effects of a metabolic disorder. This method comprises co-administering to the subject an effective amount of a dopamine receptor modulator.

An additional embodiment of the present invention is a method for counter-acting the dopamine antagonist activity of (S)-sulpiride in racemic (RS)-sulpiride administered to a subject to prevent, treat, or ameliorate the effects of a metabolic disorder. This method comprises co-administering to the subject an effective amount of a dopamine receptor modulator.

### BRIEF DESCRIPTION OF THE DRAWINGS

FIG. 1 shows that racemic amisulpride stimulates glucose disposal in diet-induced obesity (DIO) mice. DIO mice were treated once daily intraperitoneally (i.p.) for 15 days with (R/S)-amisulpride at the doses indicated, 100 mg/kg metformin, or with vehicle. Oral glucose tolerance tests (OGTTs) were performed on treated mice at day 5 (FIGS. 1a and 1b) and day 15 (FIGS. 1b and 1d). On the day of OGTT, following an overnight fast, animals were dosed 15 minutes prior to first glucose measurement and 30 minutes prior to glucose challenge. Fasting glucose levels were also collected from untreated mice that had been maintained on a normal diet (normal). The total area under the curve (AUC) for OGTTs in FIGS. 1a and 1b are represented in FIGS. 1c and 1d, respectively. All data are presented as mean±standard error of the mean (SEM) (n=6), *P<0.05.

FIG. 2 shows the dose response effect of racemic and chirally pure (R)-amisulpride on glucose disposal in high fat fed mice. DIO mice were treated once daily i.p. for 5 days with (R/S)-amisulpride (FIG. 2a), (R)-amisulpride (FIGS. 2b and 2c), and (S)-amisulpride (FIG. 2c) at the doses indicated. OGTTs were performed, as described in FIG. 1, on treated mice and glucose AUCs calculated. All data are presented as mean±SEM (n=6), *P<0.05.

FIG. 3 shows the effects of (R)-amisulpride on insulin secretion and active glucagon-like peptide-1 (GLP-1) in normal mice. Normal mice were fasted for 6 hours prior to i.p. treatment with (R)-amisulpride (10 mg/kg). An OGTT was performed with blood collected for insulin analysis. Insulin (FIG. 3a) and glucose (FIG. 3b) AUCs were calculated, across time periods indicated on the y-axis. Following OGTT, animals were washed out for 1 week prior to determination of GLP-1 levels (FIG. 3c). Mice were fasted for 6 hours prior to i.p. treatment with (R)-amisulpride (10 mg/kg). Animals were then challenged with a glucose bolus and blood collected from the hepatoportal vein for active GLP-1 measurement. All data are presented as mean±SEM (n=12), *P<0.05.

FIG. 4 shows the separation of the stereoisomers (S)(−)-amisulpride and (R)(+)-amisulpride from (R/S)-amisulpride.

FIG. 5 shows that amisulpride and a close structural relative, (R/S)-5-(aminosulfonyl)-N[1-ethylpyrrolidin-2-yl)methyl]2-methoxy-benzamide ((R/S)-sulpride), have glucose lowering effects. C57BL/6 mice were fed a high fat diet from 6 to 11 weeks of age, resulting in diet-induced obesity. Animals were then dosed i.p. with the indicated compounds once daily for 5 days (all treatments at 125 mg/kg except for metformin dosed at 100 mg/kg), After 5 days of dosing, animals were fasted overnight prior to an oral glucose tolerance test (OGTT). The morning after fasting, a baseline glucose level was measured, followed by compound or vehicle dosing. A second blood glucose level was measured 15 minutes later. At 30 minutes past the first glucose measurement, animals were challenged with a 1.5 g/kg glucose solution per os (p.o.), and blood glucose levels determined 15 and 45 minutes later. Areas under the curve were determined for the blood glucose levels, baselined at the first measurement. Significance (P<0.05; one-way ANOVA with Dunnett post-hoc comparison) compared to vehicle group is indicated by *.

FIG. 6 shows the pharmacokinetic profile of (R)-amisulpride in mice. Normal male mice were treated i.p. with 10 mg/kg (R)-amisulpride. Drug levels were determined from plasma at the times indicated (n=4 per time point).

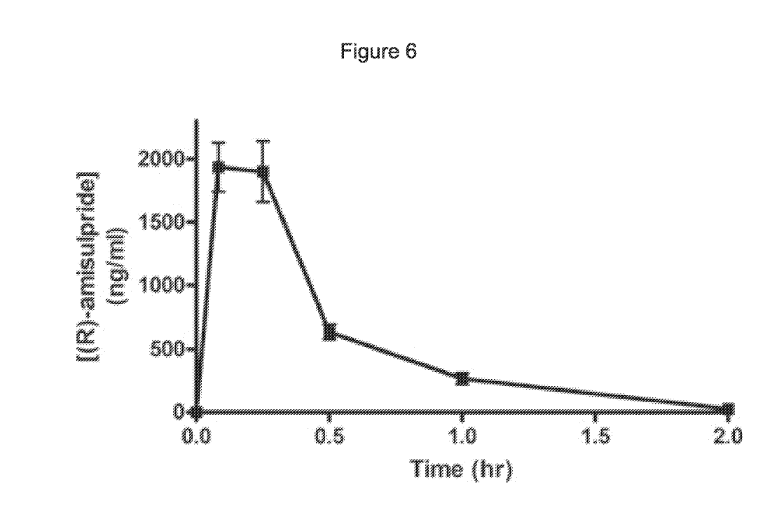

### DETAILED DESCRIPTION OF THE INVENTION

One embodiment of the present invention is a composition that comprises a pharmaceutically acceptable carrier and a therapeutically effective amount of an enantiomerically pure (R)(+)-amisulpride, an enantiomerically pure (R)(+)-sulpiride, or a pharmaceutically acceptable salt thereof.

As used herein, “enantiomerically pure” means the presence of one enantiomer, and complete absence of the other enantiomer or a presence of the other enantiomer in a concentration of at the most 10%, such as, e.g., at the most 5%, 4%, 3%, 2%, 1%, 0.5%, 0.4%, 0.3%, 0.2%, 0.1%, 0.05%.

As used herein, the term “enantiomer” refers to a member of a pair of stereoisomers whose molecules are non-superimposable mirror images of one another. The term “stereoisomer” refers to a compound made up of the same atoms bonded by the same bonds but having different three-dimensional structures which are not interchangeable. The three-dimensional structures are called configurations. The terms “racemate” or “racemic mixture” refer to a mixture of enantiomers. The term “chiral center” refers to a carbon atom to which four different groups are attached. The term “enantiomeric enrichment” as used herein refers to the increase in the amount of one enantiomer as compared to the other.

It is appreciated that compounds of the present invention having a chiral center may exist in and be isolated in racemic forms. Some compounds may exhibit polymorphism. It is well known in the art how to prepare enantiomerically pure forms of a compound, such as that described by J. Jacques, et al., “Enantiomers, Racemates, and Resolutions”, John Wiley and Sons, Inc., 1981. Examples of methods to obtain enantiomerically pure forms of a compound include at least the following:
i) physical separation of crystals—a technique whereby macroscopic crystals of the individual enantiomers are manually separated. This technique may be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually, in principle, all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent; and
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Preferably, the enantiomers are separated using high-performance liquid chromatography, as disclosed in the Examples herein. In another preferred method, chiral chromatography with a suitable organic solvent, such as ethanol/acetonitrile and Chiralpak AD packing, 20 micron may also be utilized to effect separation of the enantiomers.

Another embodiment of the present invention is a composition that comprises (i) a pharmaceutically acceptable carrier; (ii) a therapeutically effective amount of racemic (RS)-amisulpride, (RS)-sulpiride or a pharmaceutically acceptable salt thereof; and (iii) a dopamine receptor modulator.

An additional embodiment the present invention is a composition that comprises (i) a pharmaceutically acceptable carrier; (ii) a therapeutically effective amount of an enantiomerically pure (R)(+)-amisulpride, an enantiomerically pure (R)(+)-sulpiride, or a pharmaceutically acceptable salt thereof; and (iii) a dopamine receptor modulator.

In the present invention, a dopamine receptor “modulator” means a substance which can change the activity or the expression of a dopamine receptor. Dopamine receptors are a class of G protein-coupled receptors that use the neurotransmitter dopamine as the primary endogenous ligand. There are at least five subtypes of dopamine receptors: D1, D2, D3, D4, and D5. Preferably, the dopamine receptor modulator is a dopamine D2-receptor modulator. More preferably, the dopamine receptor modulator is a dopamine D2-receptor agonist.

As used herein, a “dopamine D2-receptor agonist” means a substance that activates dopamine D2 receptors in the absence of dopamine. Preferably, the dopamine D2-receptor agonist is selected from alentemol; apomorphine; biperiden; bromocriptine; cabergoline; carmoxirole; ciladopa; dopexamine; fenoldopam; ibopamine; levodopa; lisuride; methylenedioxypropylnoraporphine; naxagolide; N-allylnoraporphine; pergolide; pramipexole; propylnorapomorphine; protokylol; quinagolide; quinpirole; quinelorane, ropinirole; roxindole; talipexole; terguride; trihexyphenidyl; and trihydroxyaporphine; LY171555 (4aR-trans-4,4-a,5,6,7,8,8a,9-o-dihydro-5n-propyl-2H-pyrazolo-3-4-quinoline HCl); PPHT ((±)-2-(N-phenylethyl-N-propyl)amino-5-hydroxytetralin); or TNPA (2,10,11-trihydroxy-N-propylnoraporphine); or salts or combinations thereof. More preferably, dopamine D2-receptor agonist is bromocriptine or a pharmaceutically acceptable salt thereof.

Any of the compositions of the present invention may further comprise at least one additional active agent, which may be albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, or salts or combinations thereof. Preferably, the additional active agent increases insulin sensitivity when administered to a subject, such as metformin, pioglitazone, and rosiglitazone maleate.

An additional embodiment of this invention is a method for preventing, treating, or ameliorating the effects of a metabolic disorder or key element thereof in a subject. This method comprises administering to the subject an effective amount of any of the compositions of the present invention, including those containing additional active agents as set forth above.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject, e.g., patient, population. Accordingly, a given subject or subject, e.g., patient, population may fail to respond or respond inadequately to treatment.

As used herein, the terms “ameliorate”, “ameliorating” and grammatical variations thereof mean to decrease the severity of the symptoms of a disease in a subject.

As used herein, the terms “prevent”, “preventing” and grammatical variations thereof mean to administer a compound or a composition of the present invention to a subject who has not been diagnosed as having the disease or condition at the time of administration, but who could be expected to develop the disease or condition or be at increased risk for the disease or condition. Preventing also includes administration of at least one compound or a composition of the present invention to those subjects thought to be predisposed to the disease or condition due to age, familial history, genetic or chromosomal abnormalities, due to the presence of one or more biological markers for the disease or condition and/or due to environmental factors.

As used herein, a “metabolic disorder” means a condition in which normal chemical processes that take place in a cell or an organism to produce energy and basic materials needed for the life, such as, e.g., the formation, breakdown and interconversion of carbohydrates, are disrupted. A “key element” as used herein means a significant factor, symptom, or indication of the metabolic disorder.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

Preferably, in the present invention, the metabolic disorder or key element thereof is type 2 diabetes, prediabetes, metabolic syndrome, insulin resistance, hyperinsulinemia, cardiovascular disease, obesity, elevated plasma norepinephrine, elevated cardiovascular-related inflammatory factors or potentiators of vascular endothelial dysfunction, hyperlipoproteinemia, atherosclerosis, hyperphagia, hyperglycemia, hyperlipidemia, hypertension, or high blood pressure. In the present invention, a key element of the metabolic disorder may be exemplified by, but not limited to, the following: impaired fasting glucose, impaired glucose tolerance, increased waist circumference, increased visceral fat content, increased fasting plasma glucose, increased fasting plasma triglycerides, increased fasting plasma free fatty acids, decreased fasting plasma high density lipoprotein level, increased systolic or diastolic blood pressure, increased plasma postprandial triglyceride or free fatty acid levels, increased cellular oxidative stress or plasma indicators thereof, increased circulating hypercoagulative state, arteriosclerosis, coronary artery disease, peripheral vascular disease, congestive heart failure, renal disease including renal insufficiency, hepatic steatosis, or cerebrovascular disease.

In this embodiment, the method may further comprise administering to the subject at least one additional active agent selected from the group consisting of albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, and salts and combinations thereof.

A further embodiment of this invention is a method for modulating blood glucose levels in a subject. This method comprises administering to the subject an effective amount of any of the compositions according to the present invention, and optionally administering to the subject at least one additional active agent as previously defined. The subject may be a mammal, such as a human, a laboratory animal, a domestic animal, or an agricultural animal. Preferably, the subject is human.

Yet another embodiment of this invention is a method for preventing, treating, or ameliorating the effects of diabetes in a subject. This method comprises administering to the subject an effective amount of any of the compositions according to the present invention, and optionally administering to the subject at least one additional active agent as previously defined.

In this embodiment, the diabetes is type II diabetes, diabetes associated with genetic defects of the β-cell, diabetes resulting from genetic defects in insulin action, diabetes caused by a disease of the exocrine pancreas, diabetes caused by endocrinopathies, drug- or chemical-induced diabetes, diabetes caused by infections, immune-mediated diabetes, and gestational diabetes mellitus. In the present invention, “diabetes associated with genetic defects of the β-cell” includes mutations on chromosome 12 in a hepatic transcription factor referred to as hepatocyte nuclear factor (HNF)-1α, mutations in the glucokinase gene on chromosome 7p, mutation at position 3,243 in the tRNA leucine gene, and mutations in other transcription factors, including HNF-4α, HNF-1β, insulin promoter factor (IPF)-1, and NeuroD1. In the present invention, “diabetes resulting from genetic defects in insulin action” includes Type A insulin resistance, Leprechaunism, Rabson-Mendenhall syndrome, and lipoatrophic diabetes. In the present invention, “diabetes caused by a disease of the exocrine pancreas” includes those diseases caused by pancreatitis, trauma, infection, pancreatectomy, pancreatic carcinoma, adrenocarcinoma, cystic fibrosis, hemochromatosis, and fibrocalculous pancreatopathy. In the present invention, “diabetes caused by endocrinopathies” includes diabetes caused by acromegaly, Cushing's syndrome, glucagonoma, pheochromocytoma, hyperthyroidism, somatostatinoma, and aldosteronoma. In the present invention, “drug or chemical-induced diabetes” includes diabetes that is induced by exposure to, e.g., N-3 pyridylmethyl-N′ 4 nitrophenyl urea (Vacor), nicotinic acid, glucocorticoids, pentamidine, thyroid hormone, diazoside, β-adrenergic agonists, thiazides, dilantin, γ-interferon. In the present invention, “diabetes caused by infections,” includes diabetes caused by, e.g., congenital rubella and cytomegalovirus. “Immune-mediated diabetes” includes “stiff-man” syndrome and the production of anti-insulin receptor antibodies.

An additional embodiment of this invention is a unit dosage. This unit dosage comprises any of the compositions of the present invention.

A further embodiment of this invention is a method for counter-acting the dopamine antagonist activity of (S)-amisulpride in racemic (RS)-amisulpride administered to a subject to prevent, treat, or ameliorate the effects of a metabolic disorder. This method comprises co-administering to the subject an effective amount of a dopamine receptor modulator, such as a D2-receptor agonist as previously defined. Preferably, the dopamine D2-receptor agonist is bromocriptine or a pharmaceutically acceptable salt thereof. In this embodiment, the metabolic disorder or the key elements thereof are as defined above.

In the present invention, “co-administration” or “co-administering” means administration of two or more compounds together in the same composition, simultaneously in separate compositions, or as separate compositions administered at different times, as deemed most appropriate by a physician.

As used herein, “counter-acting” the activity of a substance means to blunt the effect of or to neutralize the effect(s) of that substance, e.g., the dopamine antagonist activity of (S)-amisulpride or (S)-sulpiride. As used herein, “dopamine antagonist activity” means the ability of a substance, such as e.g., (S)-amisulpride or (S)-sulpiride, to bind to the dopamine receptor but not activate such a receptor.

An additional embodiment of the present invention is a method for counter-acting the dopamine antagonist activity of (S)-sulpiride in racemic (RS)-sulpiride administered to a subject to prevent, treat, or ameliorate the effects of a metabolic disorder. This method comprises co-administering to the subject an effective amount of a dopamine receptor modulator, such as a D2-receptor agonist as disclosed above. Preferably, the dopamine D2-receptor agonist is bromocriptine or a pharmaceutically acceptable salt thereof. In this embodiment, the metabolic disorder or the key elements thereof are as defined above.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of sulpiride or amisulpride in the compositions disclosed herein is from about 1 mg/kg to about 2400 mg/kg per day, such as from about 1 mg/kg to about 1200 mg/kg per day, including from about 50 mg/kg to about 1200 mg/kg per day. Other representative dosages of such agents include about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of sulpiride or amisulpride in the compositions disclosed herein maybe administered as two, three, four, five, six or more sub-doses, administered separately at appropriate intervals throughout the day.

A suitable, non-limiting example of a dosage of the dopamine receptor modulator in the compositions disclosed herein is from about 0.1 to 100 mg/day, such as from about 0.5 mg/day to about 40 mg/day, including from about 1 mg/day to about 10 mg/day. Other representative dosages of such an agent include about 0.2 mg/day, 0.5 mg/day, 0.7 mg/day, 1 mg/day, 1.2 mg/day, 1.5 mg/day, 2 mg/day, 2.5 mg/day, 3 mg/day, 3.5 mg/day, 4 mg/day, 4.5 mg/day, 5 mg/day, 5.5 mg/day, 6 mg/day, 6.5 mg/day, 7 mg/day, 7.5 mg/day, 8 mg/day, 8.5 mg/day, 9 mg/day, 9.5 mg/day, 10 mg/day, 15 mg/day, 20 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, and 100 mg/day. The effective dose of the dopamine receptor modulator in the compositions disclosed herein maybe administered as two, three, four, five, six or more sub-doses, administered separately at appropriate intervals throughout the day.

A composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a composition of the present invention may be administered in conjunction with other treatments. A composition of the present invention maybe encapsulated or otherwise protected against gastric or other secretions, if desired.

The compositions of the invention comprise one or more active ingredients in admixture with one or more pharmaceutically-acceptable carriers and, optionally, one or more other compounds, drugs, ingredients and/or materials.

Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions of the present invention for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Compositions of the present invention which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods of the present invention. These examples are illustrative only and are not intended to limit the scope of the invention in any way.

### EXAMPLES

### Example 1

### Research Design and Methods

### Chemicals

Racemic amisulpride was obtained from LKT Laboratories (St. Paul, Minn.). Amisulpride enantiomers were prepared using chiral high-performance liquid chromatography (HPLC) by Chemietek (Indianapolis, Ind.), and the purity of the enantiomers was subsequently confirmed by Chemtos (Austin, Tex.).

### Diet-induced Obesity

High-fat diet feeding in rodents changes multiple biochemical and physiological parameters that reflect the biochemical and physiological changes observed in diet induced obesity (D10). Such diets induce dramatic changes in weight gain that are concomitant with elevations in serum cholesterol, lipids, and triglycerides. Moreover, these high fat diets can lead to atherosclerotic lesions as well as insulin resistance and dysregulation of glucose homeostatic mechanisms that are consistent with obesity induced changes in humans.

### Oral Glucose Tolerance Test (OGTT)

Type II diabetes is characterized by high blood glucose levels in the presence of normal amounts of insulin. The animal model or type II diabetes used in these studies involves administering high levels of glucose and then measuring blood glucose levels over time. The test is designed to determine the ability of the experimental animal to maintain glucose homeostasis over time. Drugs that lower blood glucose levels in type II diabetic patients also lower blood glucose levels in this animal model of type II diabetes.

Male C57BL/6J mice were fed a high fat diet for 5 weeks (age 6-11 weeks) prior to study. Table 1 below shows the composition of the high fat diet. DIO mice were food deprived overnight prior to glucose load (1.5 g/kg, t0). Mice were treated with drug or vehicle 30 minutes before glucose load (t-30). Blood glucose was measured with a glucometer (Accu-Chek® Roche, Indianapolis, IN), from the tip of the tail in free moving mice, at 15 minutes before (t-15), 15 minutes after (t15), and 45 minutes after (t45) glucose load. Table 1

TABLE 1

| | Gram % | Kcal % |
|---|---|---|
| Protein | 26.2 | 20 |
| Carbohydrate | 26.3 | 20 |
| Fat | 34.9 | 60 |
| Kcal/gm | 5.24 | |

Insulin and GLP1 assays

Normal male 12 week old C57BL/6J mice were food deprived 6 hours before the glucose load (1.5 g/kg, t0). Mice were treated with drug or vehicle 30 minutes before glucose load (t-30). Blood glucose was measured with a glucometer (Accu-Chek® Roche, Nutley, N.J.), from the tip of the tail in free moving mice, at t-30, t0, t15, t30, t60, t90, t120 minutes. Blood collection (about 20 μl) was performed from the tip of the tail at t-30, t15 and t30 for plasma insulin determination (ELISA kit, Alpco Diagnostics, Salem, N.H.). After a 1-week washout period, mice were randomly allocated into 2 groups. Mice were food deprived 6 hours before the glucose load (1.5 g/kg, t0) was performed. Mice were treated with drug or vehicle 30 minutes before glucose load. Blood glucose was measured at t0 and t15 minutes. At t15, mice were anesthetized with isoflurane and blood was collected from the hepatoportal vein (on EDTA/diprotin/ aprotinin anticoagulant cocktail). Plasma was rapidly prepared and stored at -80° C. The active form of GLP-1 was then measured (ELISA kit, Millipore, Billerica, Mass.).

### Prolactin Assay

Mice were treated with drug or vehicle 30 minutes before an OGTT (t-30). At 45 minutes after glucose load (t45), mice were bled via retro-orbital sinus into EDTA microfuge tubes. Plasma was rapidly prepared and stored at −80° C. until measurement of prolactin levels (ELISA kit, Genway Biotech, San Diego, Calif.).

### Pharmacokinetic Analysis

Male C57B1/6 mice (n =4, two cohorts) were treated with drug and at the designated times after dosing (5, 15, 30, 60 and 120), whole blood was collected via the retro-orbital sinus in heparanized tubes and plasma prepared by centrifugation at 1650 relative centrifugal force (RCF) at 4° C. Plasma samples were extracted by a standard protocol using an acetonitrile/protein precipitation method, and levels of test agent were analyzed by LC/MS/MS.

### Target Assays

Amisulpride was evaluated at 0.37, 1.1, 3.3, and 10 μM, using the PatchXpress 7000A (Molecular Devices LLC, Sunnyvale, Calif.), in HEK293 cells expressing Kir6.2/SUR1 potassium channels, following channel activation with 300 μM diazoxide. Glybenclamide (0.3 μM) was used as a positive control and blocked Kir_{6.2}/SUR1 current by 95%.

Amisulpride was evaluated in a human recombinant dipeptidyl dipeptidase-4 (DPP4) assay using a fluorogenic substrate, GP-AMC. DPP4 inhibitor K579 was used as a positive control. Assay was performed by Cerep (Bois l'Eveque, France).

### Data Analysis

All data are presented as means±standard error of the mean (SEM). Differences between groups were assessed by unpaired t-test or one-way analysis of variance (ANOVA) with post-hoc Dunnett's test. A P value of <0.05 was considered significant.

### Example 2

### Separation of (R)(+) Isomer of Amisulpride from (S)(−) Amisulpride

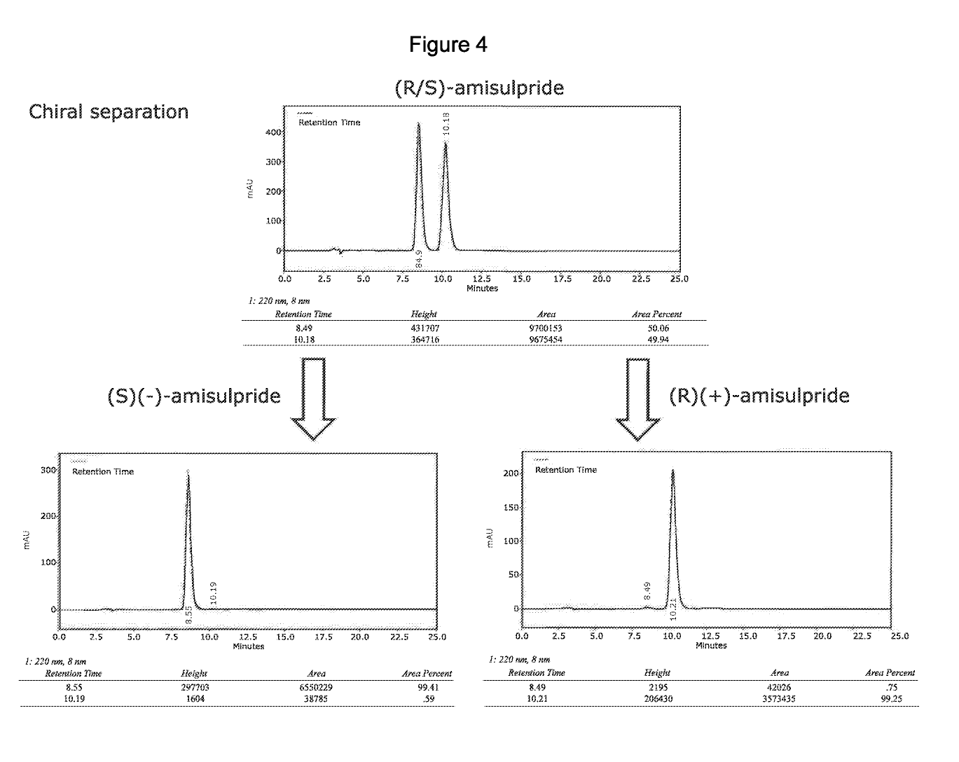

The (R)(+) isomer of amisulpride was separated from (S)(−) amisulpride using a chiral HPLC column (FIG. 4). The detailed HPLC data are shown in

Tables 2-4 below. Racemic (R/S)-amisulpride has the profile shown in Table 2 below.

TABLE 2

| Retention Time | Height | Area | Area Percent |
|---|---|---|---|
| 8.49 | 431707 | 9700153 | 50.96 |
| 10.18 | 364716 | 967584 | 49.94 |

Purified (S)(−) amisulpride has the profile shown in Table 3 below.

TABLE 3

| Retention Time | Height | Area | Area Percent |
|---|---|---|---|
| 8.55 | 297703 | 6550229 | 99.41 |
| 10.19 | 1604 | 88785 | 0.59 |

Purified (R)(+) amisulpride has the profile shown in Table 4 below.

TABLE 4

| Retention Time | Height | Area | Area Percent |
|---|---|---|---|
| 8.49 | 2195 | 42028 | 0.75 |
| 10.21 | 206430 | 5577435 | 99.25 |

### Example 3

### Effect of Amisulpride on Glucose Tolerance

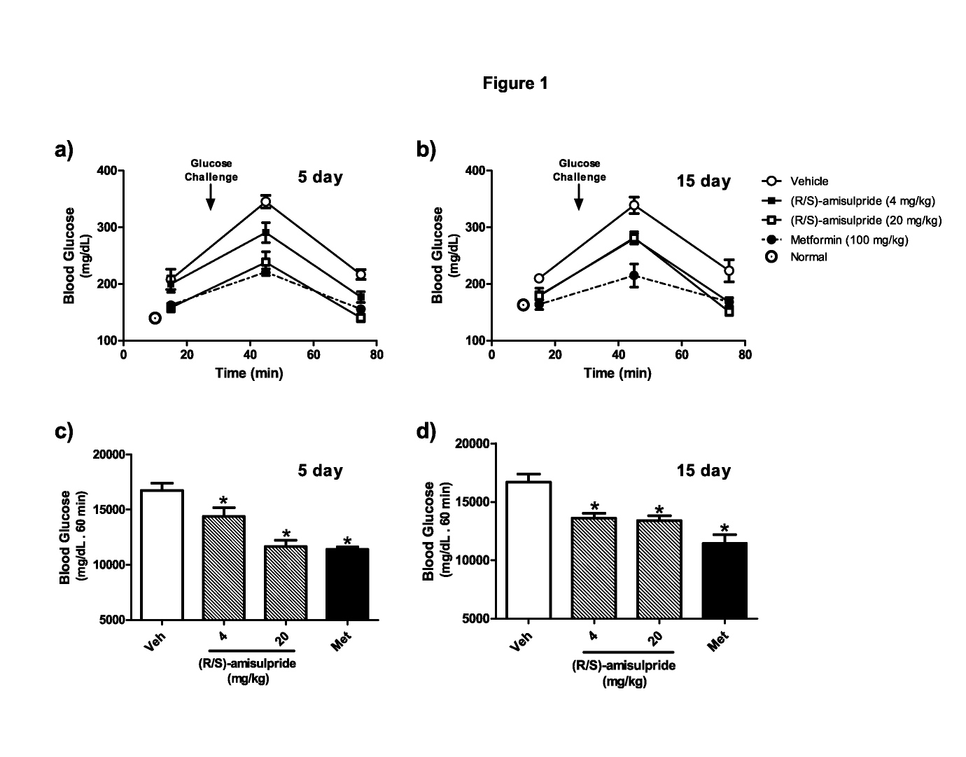

In these studies evaluating the effect of racemic amisulpride on oral glucose tolerance, diet-induced obese (D10) mice were dosed once daily for 15 days at pharmacologically relevant doses of amisulpride and an oral glucose tolerance test (OGTT) performed on days 5 and 15. A significant reduction in glucose excursion during the OGTT was observed at both amisulpride doses and both days of treatment (FIG. 1). In part, this effect may be explained by a trend in reduced fasting blood glucose levels, although, this was only significant at 20 mg/kg amisulpride following 5 days of treatment (208.2±17.9 mg/dL, in vehicle treated animal, vs. 158.7±7.5 mg/dL, in amisulpride treated animals; P<0.05). No changes in body weight were observed at either time-point or dose (data not shown).

Amisulpride is a chiral compound that is produced and prescribed in Europe as a racemic mixture. Studies of the individual isomers have indicated differences in binding affinities to D2/D3 receptors and differences in modulation of dopaminergic signaling in-vivo. In-vitro assays have demonstrated that (R)-amisulpride is 20-40 times less potent than (S)-amisulpride in displacing dopamine D2/D3 receptor ligands (12). In a rat study comparing the effect of amisulpride enantiomers on serum prolactin levels, a marker of dopamine D2 receptor blockade, (R)-amisulpride only induced a 1-fold increase in serum levels compared to a 4-fold increase with (S)-amisulpride (9). This was accompanied by a 40-fold increase in ED₅₀ of (R)-amisulpride required to reach maximal effect, compared to (S)-amisulpride (9).

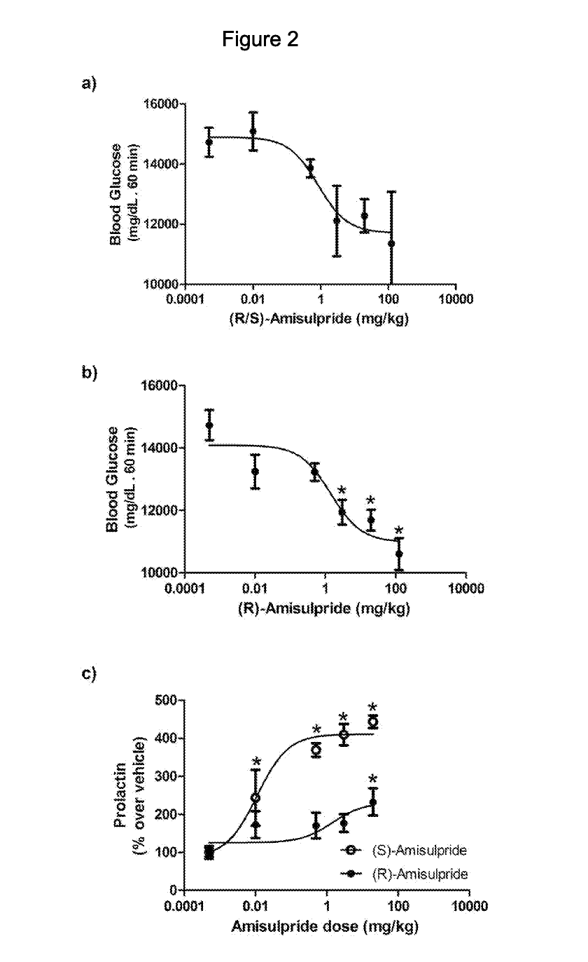

To assess the potency and enantiomer selectivity of amisulpride on glucose metabolism, the chirally pure (R) and (S) enantiomers were compared to racemic drug in the DIO mouse model. Both (R/S)-amisulpride and chirally pure forms of amisulpride were dosed once daily for 5 days in DIO mice prior to an OGTT. All compounds reduced glucose excursions in a dose-responsive manner with a calculated ED₅₀ approximating 1 mg/kg (FIGS. 2a and 2b) ((S)-amisulpride data not shown). Furthermore, the effect of R and S forms of amisulpride on circulating prolactin levels closely mirrored that reported in the rat (9), with a substantially weaker effect of the R compared to the S form (ED₅₀ of 1.5 and 0.01 mg/kg, for (R)-amisulpride and (S)-amisulpride, respectively) (FIG. 2c).

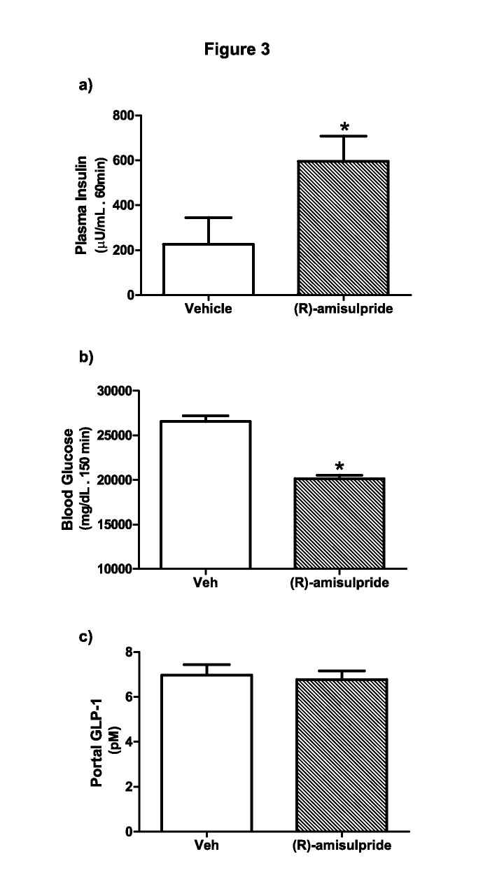

To mechanistically dissect the effect of amisulpride on glucose disposal, in-vivo studies were performed assessing drug effect on insulin secretion. An OGTT, with blood collection for insulin analysis, was performed in normal mice treated with a single dose of (R)-amisulpride. (R)-amisulpride (10 mg/kg) significantly decreased glucose excursions and increased circulating insulin levels approximately 2.5-fold, compared to vehicle (FIG. 3). Modulation of numerous GPCRs, ion channels and enzymes in the pancreatic 13-cell can result in enhanced insulin secretion, either coupled to or independently of glucose sensing (13,14). Elevating serum GLP-1 levels directly or through inhibition of dipeptidyl dipeptidase-4 (DPP4), results in glucose-stimulated insulin secretion (GSIS) (15). In the present study, active form GLP-1 levels sampled from the hepatic portal vein of normal mice remained unchanged with drug treatment (FIG. 3), as did DPP4 activity measured in-vitro (Table 5 below). Furthermore, the sulphonylurea receptor, a prototypic target for insulin secretagogues, was unaffected by amisulpride treatment in-vitro (Table 5). Pharmacokinetic analysis of (R)-amisulpride confirmed that maximal plasma drug levels did not exceed concentrations used in the in vitro assays (FIG. 4).

TABLE 5
Lack of amisulpride effect on key modulators of insulin secretion

| | (R/S)-amisulpride dose | % inhibition | |
|---|---|---|---|
| Kir6.2/SUR1 | 10 μM | 3.4 (2.4) | ns |
| DPP4 | 10 μM | 1.9 (2.0) | ns |

In Table 5 above, (R/S)-amisulpride was tested for activity in a cell-based assay of Kir_{6.2}/SUR1 and in-vitro assay of DPP4. Inhibition is represented as % of control. “ns” represents not statistically significant or P>0.05.

Deregulation of glucose homeostasis is a common feature in patients with schizophrenia who are treated with antipsychotics. While a number of factors influence the diabetes risk conferred by antipsychotic treatment, it is clear that the SGA amisulpride has a significantly less deleterious impact on glucose metabolism compared to other SGAs (7,8). These experiments assessed the actions of amisulpride on glucose metabolism in diet-induced obese mice and found a significant and pharmacologically potent anti-diabetic effect of this drug. Mechanistically, this effect is in part explained by a drug-induced enhancement of insulin-secretion.

As noted above, amisulpride is a racemic drug that antagonizes dopamine D2/D3 receptors with low nM potency (16). Pharmacological studies have demonstrated both inhibition and enhancement of dopaminergic transmission resulting from high and low doses of amisulpride, respectively (6). The beneficial effects of low dose amisulpride in depression have been attributed to enhanced dopaminergic signaling, although, an alternative hypothesis has been offered involving antagonism of the 5-HT₇ₐ receptor (17).

Bromocriptine, a dopamine D2-receptor agonist, has recently gained US marketing approval for the treatment of diabetes. At low doses, amisulpride may increase dopamine transmission by preferential antagonism of presynaptic D2/D3 receptors, and so, in providing a mechanistic explanation for the effect of amisulpride on glucose metabolism, it is first important to consider dopaminergic signaling.

Important differences in the mechanisms of glucose lowering are apparent between amisulpride and bromocriptine. Clinical studies of bromocriptine suggest insulin sensitizing actions and not enhanced insulin secretion (18). A preclinical study of bromocriptine in the ob/ob mouse demonstrated marked improvements in various metabolic parameters and decreases in circulating levels of insulin (19). Interestingly, in a study of normal mice, acute treatment with bromocriptine increases fasting glucose levels and worsens glucose tolerance, which is clearly at odds with chronic studies in insulin-resistant rodents (20). In the same study, glucose-stimulated insulin secretion (GSIS) was assessed in INS-1E cells and shown to be inhibited by bromocriptine treatment (20). Furthermore, dopamine treatment of isolated mouse islet preparations reduced in-vitro GSIS (21). These descriptions of bromocriptine action do not align with the effect of amisulpride on insulin secretion observed in the present study. Moreover, the observation that the less dopaminergically active (R) isomer of amisulpride (as demonstrated by weak effect on prolactinemia) was active in the OGTT suggests that modulation of dopamine signaling is an unlikely explanation for the glucose lowering effects seen with amisulpride. With respect to the other known activities of amisulpride against 5-HT₇ₐ or 5-HT_{2b} receptors, no data exist in the literature linking antagonism of these receptors to effects on glucose homeostasis.

Combination treatment of racemic amisulpride with bromocriptine may obviate the need for chiral separation of racemic amisulpride, and specific development of (R)-amisulpride. Dopamine agonist activity of bromocriptine is expected to cancel out the dopamine antagonist activity of (S)-amisulpride, and so maintain serum levels of prolactin in a normal range. Relieving the diabetogenic effect of (S)-amisulpride would result in stronger anti-diabetic actions of racemic amisulpride. A further benefit of this combination is that bromocriptine and amisulpride exhibit different mechanisms in inducing glucose lowering. Bromocriptine is reported to enhance insulin sensitivity while we have demonstrated amisulpride enhances insulin secretion. Combining these differing mechanistic actions is expected to result in a novel and an enhanced anti-diabetic profile.

The finding that amisulpride caused robust insulin secretion in the present study is rational given the acute and pronounced drug effect on glucose disposal, however, the molecular target of amisulpride underpinning this effect remains undetermined. A plethora of secreted factors, GPCRs and enzymes control insulin secretion. Increasing GLP-1 levels, through DPP4 inhibition, and sulphonylurea receptor modulation are two approaches that are currently used in the treatment of patients with Type II diabetes. Amisulpride had no effect on these processes.

Recently, clinical studies have suggested that high prolactin levels may contribute to insulin resistance and deregulation of glucose metabolism (23,24). It is possible that hyperprolactinemia resulting from treatment with racemic amisulpride offsets the anti-diabetic actions of this drug. If this is indeed the case, the use of (R)-amisulpride, which exhibits less impact on prolactin levels, as a treatment for diabetes is even more advantageous.

### Example 4

### Effect of Close Structural Relatives of Amisulpride on Glucose Tolerance

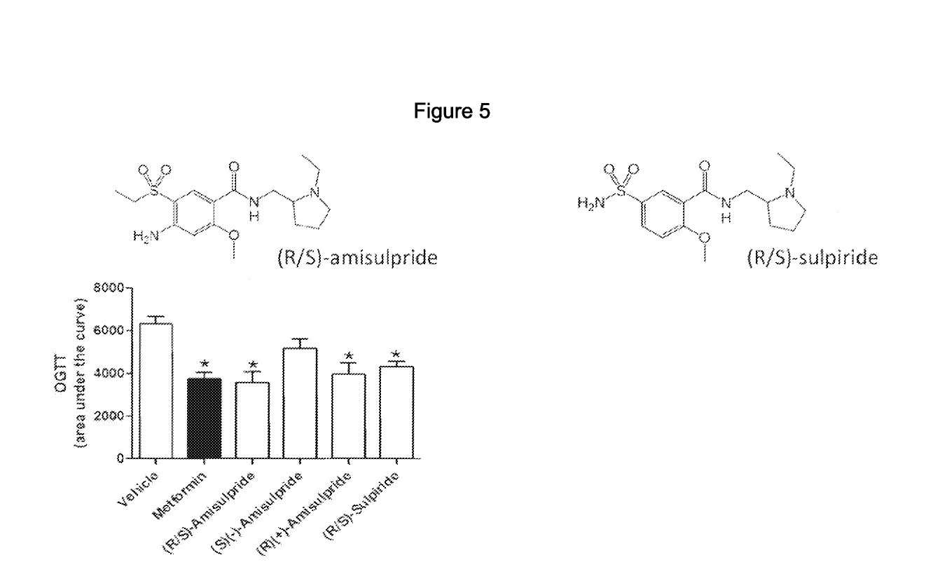

In these studies, C57BL/6 mice were fed a high fat diet from 6 to 11 weeks of age, resulting in diet-induced obesity. The animals were then dosed i.p. with (R/S) amisulpride, (S)(−) amisulpride, (R)(+) amisulpride, metformin, and (R/S)-5-(aminosulfonyl)-N[1-ethylpyrrolidin-2-yl)methyl]2-methoxy-benzamide ((R/S)-sulpride)—once daily for 5 days. All treatments were dosed at 125 mg/kg, except for metformin, which was dosed at 100 mg/kg. After 5 days of dosing, animals were fasted overnight prior to an oral glucose tolerance test (OGTT). The morning after fasting, a baseline glucose level was measured, followed by compound or vehicle dosing. A second blood glucose level was measured 15 minutes later. At 30 minutes past the first glucose measurement, animals were challenged with a 1.5 g/kg glucose solution per os (p.o.), and blood glucose levels determined 15 and 45 minutes later. Areas under the curve were determined for the blood glucose levels, baselined at the first measurement. The results showed that amisulpride and a close structural relative, (R/S)-sulpride, have glucose lowering effects (FIG. 5).

While the preferred embodiments of the invention have been described above, it will be recognized and understood that various modifications can be made in the invention and the appended claims are intended to cover all such modifications which may fall within the spirit and scope of the invention.

### DOCUMENTS

1. Buse J B, Cavazzoni P, Hornbuckle K, et al. A retrospective cohort study of diabetes mellitus and antipsychotic treatment in the United States. J Clin Epidemiol 2003;56:164-170
2. Scheen A J, De Hert M A. Abnormal glucose metabolism in patients treated with antipsychotics. Diabetes Metab 2007;33:169-175
3. Tschoner A, Engl J, Laimer M, et al. Metabolic side effects of antipsychotic medication. Int J Clin Pract 2007;61:1356-1370
4. Holt RI, Peveler RC. Association between antipsychotic drugs and diabetes. Diabetes Obes Metab 2006;8:125-135
5. Tschoner A, Engl J, Rettenbacher M, et al. Effects of six second generation antipsychotics on body weight and metabolism-risk assessment and results from a prospective study. Pharmacopsychiatry 2009;42:29-34
6. McKeage K, Plosker GL. Amisulpride: a review of its use in the management of schizophrenia. CNS Drugs 2004;18:933-956
7. Rettenbacher MA, Hummer M, Hofer A, et al. Alterations of glucose metabolism during treatment with clozapine or amisulpride: results from a prospective 16-week study. J Psychopharmacol 2007;21:400-404
8. Lin CC, Bai Y M, Wang Y C, et al. Improved body weight and metabolic outcomes in overweight or obese psychiatric patients switched to amisulpride from other atypical antipsychotics. J Clin Psychopharmacol 2009;29:529-536
9. Marchese G, Ruiu S, Casti P, et al. Effect of the amisulpride isomers on rat prolactinemia. Eur J Pharmacol 2002;448:263-266
10. Kopecek M, Bares M, Svarc J, et al. Hyperprolactinemia after low dose of amisulpride. Neuro Endocrinol Lett 2004;25:419-422
11. Juruena MF, de Sena EP, de Oliveira IR. Safety and tolerability of antipsychotics: focus on amisulpride. Drug Healthc Patient Saf 2010;2:205-211
12. Castelli MP, Mocci I, Sanna AM, et al. (−)S amisulpride binds with high affinity to cloned dopamine D(3) and D(2) receptors. Eur J Pharmacol 2001;432:143-147
13. Seino S, Shibasaki T, Minami K. Pancreatic beta-cell signaling: toward better understanding of diabetes and its treatment. Proc Jpn Acad Ser B Phys Biol Sci 2010;86:563-577
14. Ahren B. Islet G protein-coupled receptors as potential targets for treatment of type 2 diabetes. Nat Rev Drug Discov 2009;8:369-385
15. Davidson JA. Advances in therapy for type 2 diabetes: GLP-1 receptor agonists and DPP-4 inhibitors. Cleve Clin J Med 2009;76 Suppl 5:S28-38
16. Schoemaker H, Claustre Y, Fage D, et al. Neurochemical characteristics of amisulpride, an atypical dopamine D2/D3 receptor antagonist with both presynaptic and limbic selectivity. J Pharmacol Exp Ther 1997;280:83-97
17. Abbas A I, Hedlund P B, Huang X P, et al. Amisulpride is a potent 5-HT7 antagonist: relevance for antidepressant actions in vivo. Psychopharmacology (Berl) 2009;205:119-128
18. Defronzo RA. Bromocriptine: a sympatholytic, d2-dopamine agonist for the treatment of type 2 diabetes. Diabetes Care 2011;34:789-794
19. Cincotta A H, Tozzo E, Scislowski P W. Bromocriptine/SKF38393 treatment ameliorates obesity and associated metabolic dysfunctions in obese (ob/ob) mice. Life Sci 1997;61:951-956
20. de Leeuw van Weenen J E, Parlevliet E T, Maechler P, et al. The dopamine receptor D2 agonist bromocriptine inhibits glucose-stimulated insulin secretion by direct activation of the alpha2-adrenergic receptors in beta cells. Biochem Pharmacol 2010;79:1827-1836
21. Garcia-Tornadu I, Ornstein A M, Chamson-Reig A, et al. Disruption of the dopamine d2 receptor impairs insulin secretion and causes glucose intolerance. Endocrinology 2010;151:1441-1450
22. Peuskens J, De Hert M, Mortimer A. Metabolic control in patients with schizophrenia treated with amisulpride or olanzapine. Int Clin Psychopharmacol 2007;22:145-152
23. Berinder K, Nystrom T, Hoybye C, et al. Insulin sensitivity and lipid profile in prolactinoma patients before and after normalization of prolactin by dopamine agonist therapy. Pituitary 2011;14:199-207
24. Tuzcu A, Yalaki S, Arikan S, et al. Evaluation of insulin sensitivity in hyperprolactinemic subjects by euglycemic hyperinsulinemic clamp technique. Pituitary 2009;12:330-334

All documents cited in this application are hereby incorporated by reference as if recited in full herein.

What is claimed is:

1. A composition comprising a pharmaceutically acceptable carrier and a therapeutically effective amount of an enantiomerically pure (R)(+)-amisulpride, an enantiomerically pure (R)(+)-sulpiride, or a pharmaceutically acceptable salt thereof.

2. A composition comprising (i) a pharmaceutically acceptable carrier; (ii) a therapeutically effective amount of racemic (RS)-amisulpride, (RS)-sulpiride or a pharmaceutically acceptable salt thereof; and (iii) a dopamine receptor modulator.

3. A composition comprising (i) a pharmaceutically acceptable carrier; (ii) a therapeutically effective amount of an enantiomerically pure (R)(+)-amisulpride, an enantiomerically pure (R)(+)-sulpiride, or a pharmaceutically acceptable salt thereof;
and (iii) a dopamine receptor modulator.

4. The composition according to any one of claim 2 or 3, wherein the dopamine receptor modulator is a dopamine D2-receptor agonist.

5. The composition according to claim 4, wherein the dopamine D2-receptor agonist is selected from the group consisting of alentemol; apomorphine; biperiden;
bromocriptine; cabergoline; carmoxirole; ciladopa; dopexamine; fenoldopam;
ibopamine; levodopa; lisuride; methylenedioxypropylnoraporphine; naxagolide; N-allylnoraporphine; pergolide; pramipexole; propylnorapomorphine; protokylol;
quinagolide; quinpirole; quinelorane, ropinirole; roxindole; talipexole; terguride;
trihexyphenidyl; and trihydroxyaporphine; LY171555 (4aR-trans-4,4-a,5,6,7,8,8a,9-o-dihydro-5n-propyl-2H-pyrazolo-3-4-quinoline HCl); PPHT ((±)-2-(N-phenylethyl-N-propyl)amino-5-hydroxytetralin); and TNPA (2,10,11-trihydroxy-N-propylnoraporphine); and salts and combinations thereof.

6. The composition according to claim 5, wherein the dopamine D2-receptor agonist is bromocriptine or a pharmaceutically acceptable salt thereof.

7. The composition according to any one of claim 1, 2, or 3, further comprising at least one additional active agent selected from the group consisting of albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, and salts and combinations thereof.

8. A method for preventing, treating, or ameliorating the effects of a metabolic disorder or key element thereof in a subject comprising administering to a subject an effective amount of the composition according to any one of claim 1, 2, or 3.

9. The method according to claim 8, wherein the metabolic disorder or key element thereof is selected from the group consisting of type 2 diabetes, prediabetes, metabolic syndrome, insulin resistance, hyperinsulinemia, cardiovascular disease, obesity, elevated plasma norepinephrine, elevated cardiovascular-related inflammatory factors or potentiators of vascular endothelial dysfunction, hyperlipoproteinemia, atherosclerosis, hyperphagia, hyperglycemia, hyperlipidemia, hypertension, and high blood pressure.

10. The method according to claim 8, wherein the key element of the metabolic disorder is selected from the group consisting of impaired fasting glucose, impaired glucose tolerance, increased waist circumference, increased visceral fat content, increased fasting plasma glucose, increased fasting plasma triglycerides, increased fasting plasma free fatty acids, decreased fasting plasma high density lipoprotein level, increased systolic or diastolic blood pressure, increased plasma postprandial triglyceride or free fatty acid levels, increased cellular oxidative stress or plasma indicators thereof, increased circulating hypercoagulative state, arteriosclerosis, coronary artery disease, peripheral vascular disease, congestive heart failure, renal disease including renal insufficiency, hepatic steatosis and cerebrovascular disease.

11. The method according to claim 8, further comprising administering to the subject at least one additional active agent selected from the group consisting of albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, and salts and combinations thereof.

12. A method for modulating blood glucose levels in a subject comprising administering to a subject an effective amount of the composition according to any one of claim 1, 2, or 3.

13. The method according to claim 12 further comprising administering to the subject at least one additional active agent selected from the group consisting of albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, and salts and combinations thereof.

14. A method for preventing, treating, or ameliorating the effects of diabetes in a subject comprising administering to a subject an effective amount of the composition according to any one of claim 1, 2, or 3.

15. The method according to claim 14, wherein the diabetes is selected from the group consisting of type II diabetes, diabetes associated with genetic defects of the β-cell, diabetes resulting from genetic defects in insulin action, diabetes caused by a disease of the exocrine pancreas, diabetes caused by endocrinopathies, drug- or chemical-induced diabetes, diabetes caused by infections, immune-mediated diabetes, and gestational diabetes mellitus.

16. The method according to claim 14 further comprising administering to the subject at least one additional active agent selected from the group consisting of albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, glyburide, HL-002 (HanAll Biopharma), insulin, insulin analogue (Eli Lilly), linagliptin, liraglutide, metformin, miglitol, mitiglinide, nateglinide, pioglitazone, pramlintide, repaglinide, rosiglitazone maleate, saxagliptin, sitagliptin, tolazamide, tolbutamide, vildagliptin, voglibose, and salts and combinations thereof.

17. A unit dosage comprising the composition according to any one of claim 1, 2, or 3.

18. A method for counter-acting the dopamine antagonist activity of (S)-amisulpride in racemic (RS)-amisulpride administered to a subject to prevent, treat, or ameliorate the effects of a metabolic disorder comprising co-administering to the subject an effective amount of a dopamine receptor modulator.

19. The method according to claim 18, wherein the metabolic disorder or key element thereof is selected from the group consisting of type 2 diabetes, prediabetes, metabolic syndrome, insulin resistance, hyperinsulinemia, cardiovascular disease, obesity, elevated plasma norepinephrine, elevated cardiovascular-related inflammatory factors or potentiators of vascular endothelial dysfunction, hyperlipoproteinemia, atherosclerosis, hyperphagia, hyperglycemia, hyperlipidemia, hypertension, and high blood pressure.

20. The method according to claim 18, wherein the key element of the metabolic disorder is selected from the group consisting of impaired fasting glucose, impaired glucose tolerance, increased waist circumference, increased visceral fat content, increased fasting plasma glucose, increased fasting plasma triglycerides, increased fasting plasma free fatty acids, decreased fasting plasma high density lipoprotein level, increased systolic or diastolic blood pressure, increased plasma postprandial triglyceride or free fatty acid levels, increased cellular oxidative stress or plasma indicators thereof, increased circulating hypercoagulative state, arteriosclerosis, coronary artery disease, peripheral vascular disease, congestive heart failure, renal disease including renal insufficiency, hepatic steatosis and cerebrovascular disease.

21. A method for counter-acting the dopamine antagonist activity of (S)-sulpiride in racemic (RS)-sulpiride administered to a subject to prevent, treat, or ameliorate the effects of a metabolic disorder comprising co-administering to the subject an effective amount of a dopamine receptor modulator.

22. The method according to claim 21, wherein the metabolic disorder or key element thereof is selected from the group consisting of type 2 diabetes, prediabetes, metabolic syndrome, insulin resistance, hyperinsulinemia, cardiovascular disease, obesity, elevated plasma norepinephrine, elevated cardiovascular-related inflammatory factors or potentiators of vascular endothelial dysfunction, hyperlipoproteinemia, atherosclerosis, hyperphagia, hyperglycemia, hyperlipidemia, hypertension, and high blood pressure.

23. The method according to claim 21, wherein the key element of the metabolic disorder is selected from the group consisting of impaired fasting glucose, impaired glucose tolerance, increased waist circumference, increased visceral fat content, increased fasting plasma glucose, increased fasting plasma triglycerides, increased fasting plasma free fatty acids, decreased fasting plasma high density lipoprotein level, increased systolic or diastolic blood pressure, increased plasma postprandial triglyceride or free fatty acid levels, increased cellular oxidative stress or plasma indicators thereof, increased circulating hypercoagulative state, arteriosclerosis, coronary artery disease, peripheral vascular disease, congestive heart failure, renal disease including renal insufficiency, hepatic steatosis and cerebrovascular disease.

24. The method according any one of claims 18-23, wherein the dopamine receptor modulator is a D2-receptor agonist.

25. The method according to claim 24, wherein the dopamine D2-receptor agonist is selected from the group consisting of alentemol; apomorphine; biperiden;
bromocriptine; cabergoline; carmoxirole; ciladopa; dopexamine; fenoldopam;
ibopamine; levodopa; lisuride; methylenedioxypropylnoraporphine; naxagolide; N-allylnoraporphine; pergolide; pramipexole; propylnorapomorphine; protokylol;
quinagolide; quinpirole; quinelorane, ropinirole; roxindole; talipexole; terguride;
trihexyphenidyl; and trihydroxyaporphine; LY171555 (4aR-trans-4,4-a,5,6,7,8,8a,9-o-dihydro-5n-propyl-2H-pyrazolo-3-4-quinoline HCl); PPHT ((±)-2-(N-phenylethyl-N-propyl)amino-5-hydroxytetralin); and TNPA (2,10,11-trihydroxy-N-propylnoraporphine); and salts and combinations thereof.

26. The method according to claim 25, wherein the dopamine D2-receptor agonist is bromocriptine or a pharmaceutically acceptable salt thereof.

27. The method according to any one of claim 18 or 21, wherein the subject is a mammal.

28. The method according to claim 27, wherein the mammal is selected from the group consisting of a human, a laboratory animal, a domestic animal, and an agricultural animal.

29. The method according to claim 27, wherein the subject is a human.
